(19) United States
(12) Patent Application Publication
(10) Pub. No.: US 2009/0024209 A1
(43) Pub. Date: Jan. 22, 2009

(54) Hypotubes for Intravascular Drug Delivery

(75) Inventors: Feridun Ozdil, Santa Rosa, CA (US); Joseph Berglund, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/780,702

(22) Filed: Jul. 20, 2007

(51) Int. Cl. A61F 2/06

(52) U.S. Cl. 623/1.42

(57) ABSTRACT

An implantable device capable of delivering drugs is disclosed. An example of the device is a stent that comprises at least one hypotube having a lumen and one or more pores. The lumen of the hypotube is configured to retain drugs that can be eluted through the one or more pores after deployment at a treatment site.

### FIELD OF THE INVENTION

The present invention relates to drug-eluting implantable devices for intravascular drug delivery.

### BACKGROUND OF THE INVENTION

Stenosis is the narrowing of an anatomical passageway or opening in the body, such as seen in blood vessels. A number of physiological complications have been associated with stenosis, such as ischemia, cardiomyopathy, angina pectoris, and myocardial infarction. In response, several procedures have been developed for treating stenosis. For example, in percutaneous transluminal coronary angioplasty (PTCA), a balloon catheter is inserted into a blocked or narrowed coronary blood vessel of a patient. Once the balloon is positioned at the blockage or narrowing, the balloon is inflated causing dilation of the vessel. The catheter is then removed from the site to allow blood to more freely flow through the less restricted vessel.

While the PTCA procedure has proven successful in treating stenosis in the past, several shortcomings associated with the procedure have been identified. For example, an ongoing problem with PTCA is that in about one-third of cases, the blockage or narrowing of the vessel returns often within about six months of initial treatment. It is thought that the mechanism of this “relapse,” called “restenosis,” is not solely the progression of coronary artery disease, but rather the body's immune system response to the “injury” caused by the procedure. For instance, PTCA often triggers blood clotting (i.e., “thrombosis”) at the site of the procedure resulting in renarrowing of the vessel. In addition, tissue growth at the site of treatment caused by an immune system response in the area also can occur and result in renarrowing of the vessel. This tissue growth—a migration and proliferation of the smooth muscle cells that are normally found in the media portion of the blood vessel (i.e., neointimal hyperplasia)—tends to occur during the first three to six months after the PTCA procedure, and it is often thought of as resulting from “over exuberant” tissue healing and cellular regeneration after the PTCA procedure.

Stents and/or drug therapies, either alone or in combination with the PTCA procedure, are often used to avoid or mitigate the effects or occurrence of restenosis. In general, stents are mechanical scaffoldings which may be inserted into a blocked or narrowed region of a passageway to provide and maintain its patency. During implantation, a stent can be positioned on a delivery device (for example and without limitation a balloon catheter) and advanced from an external location to an area of passageway blockage or narrowing within the body of the patient. Once positioned, the delivery device can be actuated to deploy the radially expandable stent. Expansion of the stent can result in the application of force against the internal wall of the passageway, thereby improving the patency of the passageway. Thereafter, the delivery device can be removed from the patient's body.

Stents may be manufactured in a variety of lengths and diameters and from a variety of materials ranging from metallic materials to polymers. Stents may also incorporate and release drugs (i.e., “drug-eluting stents”) that can affect endothelialization as well as the formation of and treatment of existing plaque and/or blood clots. In some instances then, drug-eluting stents can reduce, or in some cases, eliminate, the incidence of endothelialization, thrombosis and/or restenosis.

Most drug-eluting stents generally carry and release drugs in polymer matrices, such as, without limitation, silicone, polyurethane, polyvinyl alcohol, polyethylene, polyesters, hydrogels, hyaluronate, and various copolymers or blended mixtures thereof. The drug-containing polymer matrix generally is applied to the surfaces of the stent during or after its manufacture thereby forming one or more layers of stent coatings that elute the carried drug(s) once implanted at a treatment site. Thus, positioning the drug-eluting stent at a target site enables localized delivery of the drugs to the target site while providing radial support to its structure.

Although drug-eluting polymer stent coatings can be beneficial for the treatment of stenosis or restenosis, they suffer from several limitations. For example, the maximum polymer coating thickness is generally limited to about 10 to 50 microns. Therefore, the effective amount and duration of drug release is limited to the amount of drug(s) that can be included within the particular thickness of a coating.

In light of the foregoing, there is an ongoing need for improved implantable devices such as stents that are capable of both providing sufficient radially expanding force to a passageway while delivering drugs. The present invention addresses these needs, among others.

### BRIEF SUMMARY OF THE INVENTION

The present invention provides improved drug-eluting implantable devices such as stents for intravascular drug delivery. The present invention provides implantable devices with one or more generally elongate tubes (referred to herein as “hypotubes”) that can elute drugs through either the walls of the tubes (i.e., diffusive transport) and/or one or more openings or pores (hereinafter “pores”) on the tube. As such, the hypotubes allow for controlled delivery of drugs.

In one embodiment of the present invention, an implantable device for delivering a drug to a treatment site is provided comprising a hypotube, the hypotube having a lumen; and at least one drug disposed within the lumen of the hypotube, wherein the at least one drug can elute from the lumen of the hypotube through one or more pores in the hypotube.

In another embodiment, one or more of the pores are covered or plugged with a biodegradable material. In another embodiment, the hypotube is formed from a biodegradable material. In another embodiment, the biodegradable material is a material selected from the group consisting of biodegradable metals, metal alloys and polymers. In another embodiment, the biodegradable polymer is selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, and combinations thereof.

In another embodiment of the present invention, the hypotube is formed from a non-erodable polymeric material selected from the group consisting of polyether sulfone, polyamide, polycarbonate, polypropylene, high molecular weight polyethylene, polydimethylsiolxane, poly(ethylene-vinylacetate), acrylate based polymers or copolymers, polyvinyl pyrrolidinone, fluorinated polymers, and cellulose esters.

In another embodiment, the implantable device is a stent.

In another embodiment of the present invention, the lumen contains at least two compartments. In another embodiment, each of the compartments contains different drugs. In another embodiment, each of the compartments exhibits different drug release profiles. In another embodiment, the hypotube contains more than one pore and the pores are spaced along the hypotube to create different drug release profiles at different portions of the implantable device. In another embodiment, the majority of the pores are present on the proximal portion of the implantable device. In another embodiment, the majority of said pores are present on the distal portion of the implantable device. In another embodiment, the majority of the pores are present on the blood vessel lumen facing portion of the implantable device. In another embodiment, the majority of the pores are present on the ablumenal facing portion of the implantable device. In another embodiment, one compartment of the hypotube contains pores which face the blood vessel lumen while another compartment of the hypotube contains pores which face the blood vessel wall such that different drugs are delivered to the blood stream and the vascular wall.

In another embodiment of the present invention, the implantable device defines a channel and the majority of the pores are present on the portion of the hypotube contacting the channel. In another embodiment, the implantable device defines a channel and the majority of the pores are present on the portion of the hypotube that is generally opposite of the portion of the hypotube contacting the channel.

In another embodiment of the present invention, the hypotube is in a configuration selected from the group consisting of a helical configuration, a braided configuration, a mesh configuration and a woven configuration. In another embodiment, the implantable device comprises more than one hypotube. In another embodiment, the stent comprises two or more hypotubes in a configuration selected from the group consisting of a helical configuration, a braided configuration, a mesh configuration and a woven configuration.

In another embodiment, the at least one drug is combined with a biocompatible carrier before the drug is disposed within the lumen of the hypotube. In another embodiment, the biocompatible carrier comprises a biodegradable material selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, and combinations thereof.

In another embodiment of the present invention, the at least one drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. In another embodiment, the at least one drug is selected from the group consisting of sirolimus (rapamycin), tacrolimus (FK506), everolimus (certican), temsirolimus (CCI-779) and zotarolimus (ABT-578).

In one embodiment of the present invention, an implantable device for delivering a drug to a treatment site is provided comprising a biodegradable hypotube, the hypotube having a lumen; and at least one drug disposed within the lumen of the hypotube, wherein the at least one drug is released from the lumen upon degradation of the biodegradable hypotube.

Other objects, features, and advantages of the present invention will become apparent from a consideration of the following detailed description.

### BRIEF DESCRIPTION OF THE DRAWINGS

FIG. 1 depicts a perspective and partial-section (to the right of line S) view showing portions of an implantable device in accordance with an embodiment of the present invention.

FIGS. 2A and 2B depicts cross-section views of an exemplary stent from two perspectives, crosswise (FIG. 2A) and lengthwise (FIG. 2B), of an implantable device in accordance with an embodiment of the present invention.

FIG. 3 depicts an implantable device, such as stent, in accordance with an embodiment of the present invention.

FIG. 4 depicts an implantable device in accordance with another embodiment of the present invention.

### DEFINITION OF TERMS

Animal: As used herein, “animal” shall include mammals, fish, reptiles and birds. Mammals include, but are not limited to, primates (including, without limitation, humans), rodents, dogs, cats, goats, sheep, rabbits, pigs, horses and cows.

Biocompatible: As used herein, a “biocompatible” material shall include any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein, a “biodegradable” material refers to a material that is biocompatible and subject to being broken down in vivo through the action of normal biochemical pathways. From time-to-time bioerodable, bioabsorbable and biodegradable may be used interchangeably. The biodegradable polymers of the present invention are capable of being cleaved into biocompatible byproducts through hydrolytic, chemical- or enzyme-catalyzed mechanisms.

Biostable: As used herein, a “biostable” material refers to a material that is not degraded, resorbed or eroded upon exposure to body tissues, including body fluids.

Drug(s): As used herein, “drug” shall include any compound or bioactive agent having a therapeutic effect in an animal. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in the present invention.

Hypotube: As used herein, “hypotube” shall refer generally to a hollow elongate tube having at least one opening or pore within the walls of the tube.

Therapeutic effect: As used herein, “therapeutic effect” means an effect resulting from treatment of an animal that alters (e.g., improves or ameliorates) the symptoms of a disease or condition, or the structure or function of the body of the animal; or that cures a disease or condition.

### DETAILED DESCRIPTION OF THE INVENTION

The present invention provided biodegradable drug-eluting implantable devices for intravascular drug delivery. The present invention provides this advance by providing implantable devices, including stents, that comprise one or more tubes (referred to herein as “hypotubes”) within or around the structure of the device. These hypotubes contain one or more drugs that can elute drugs through either the walls of the tubes (i.e., diffusive transport) and/or one or more openings or pores (hereinafter “pores”) on the tube.

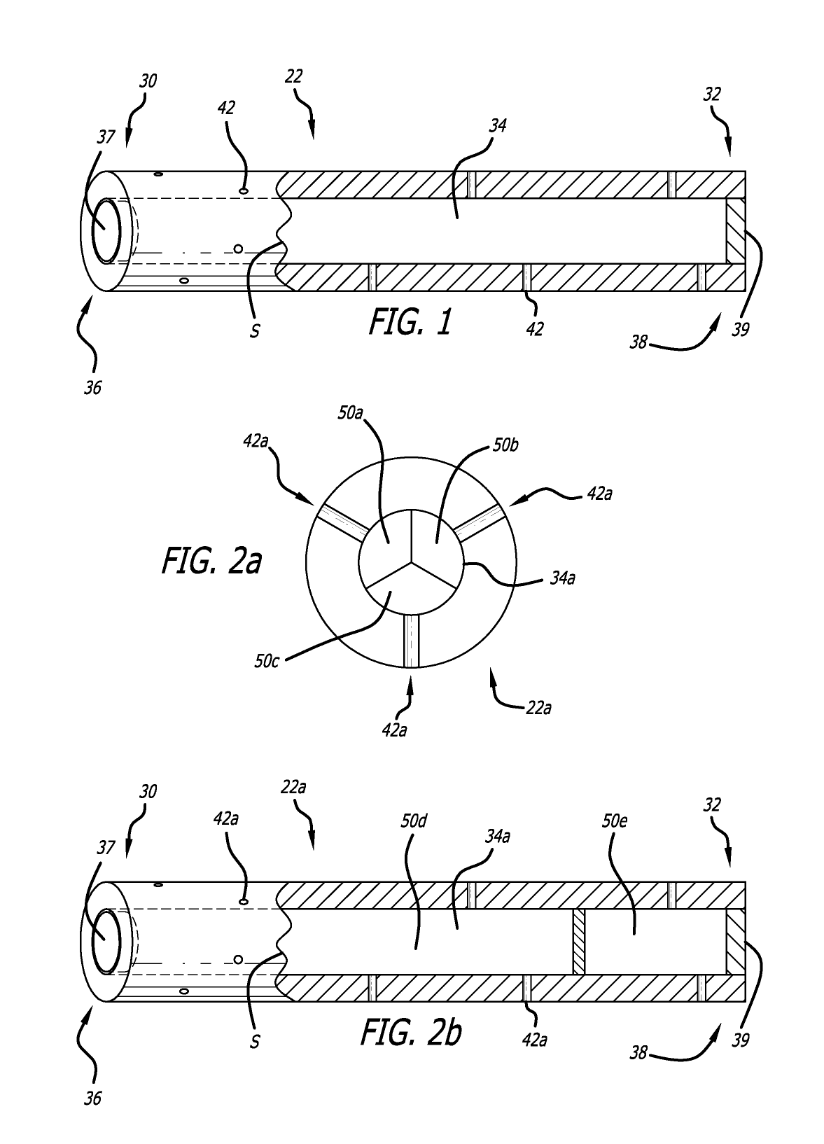

Referring to FIG. 1, a hypotube adopting aspects of the present invention is described. As shown in FIG. 1, hypotube 22 has a proximal end 30 and a distal end 32. As shown in the cross-section view of FIG. 1 (to the right of line S), hypotube 22 also has a lumen 34 extending between proximal end 30 and distal end 32. In one embodiment, hypotube 22 also comprises proximal opening 36 and distal opening 38, each of which can be in fluid communication with lumen 34. In one embodiment, one or more pores 42 formed on hypotube 22 are in fluid communication with lumen 34, as shown by the cross-section view of FIG. 1. Pores 42 are formed, for example and without limitation, using an excimer laser to achieve the preferred diameter and depth. Further, pores 42 can comprise any appropriate shape, for example and without limitation, circular, ellipitical or rectangular configurations.

As shown in FIG. 1, distal opening 38 can be covered or plugged, for example, using weld 39, or another appropriate means for covering or plugging the opening. One or more drugs can be loaded into lumen 34 through proximal opening 36, for example, using a syringe or any other suitable means. In another embodiment, proximal opening 36 can be covered or plugged, for example, using weld 37, or another appropriate means for covering or plugging the opening. One or more drugs can also be loaded into hypotube 22 through one or more pores 42 as appropriate or by other means which will be apparent to one of ordinary skill in the art. Distal opening 38 and proximal opening 36 can be covered or plugged with a biodegradable or biostable material.

In one embodiment, one or more drugs elute through one or more pores 42. In another embodiment, one or more pores 42, the distal opening 38, and/or the proximal opening 36, can initially be covered or plugged with a biocompatible material that can biodegrade or bioerode over time allowing freer drug elution over time. To further affect drug release, varying thicknesses of the biocompatible biodegradable or bioerodable material can be used to cover or plug the one or more pores 42, the distal opening 38, and/or the proximal opening 36. In one non-limiting example, hypotube 22 is coated with one or more layers of biocompatible material to cover or plug the one or more pores 42, the distal opening 38, and/or the proximal opening 36, and the one or more layers of biocompatible biodegradable material can biodegrade, bioerode, and/or otherwise dissociate from hypotube 22 to allow for drug release through the one or more pores 42, the distal opening 38, and/or the proximal opening 36 of hypotube 22.

In one embodiment, distal opening 38 and proximal opening 36 are covered or plugged with a biostable material and one or more pores 42 are covered or plugged with a biodegradable material.

In one embodiment, one or more drugs can be combined with a carrier, such as a biocompatible polymer to alter the release profile of the drug. The carrier can biodegrade or bioerode over a period of time to allow drug-elution to occur more freely over time. In another specific, non-limiting example, the carrier is generally nonbiodegradable, or biostable, that can allow drug to separate from the carrier over time (e.g., via diffusion) for controlled drug delivery.

It is contemplated that drug and/or drug/carrier can be in a variety of physical forms, including and without limitation, liquid, solid, gel and combinations thereof, when they are loaded into lumen 34 of hypotube 22. Accordingly, in some embodiments (e.g., when drug and/or drug/carrier are in a liquid form), it may be necessary to cover or plug one or more pores 42, the distal opening 38, and/or the proximal opening 36, before and/or after the drug and/or drug/carrier are loaded into lumen 34 to retain the drug and/or drug/carrier within lumen 34 for a specific amount of time (e.g., until after its deployment to a treatment site).

Further, in accordance with the present invention, any number of drug and/or drug/carrier combinations are envisioned and it is not intended that merely one or two different drugs and/or drug/carrier be employed.

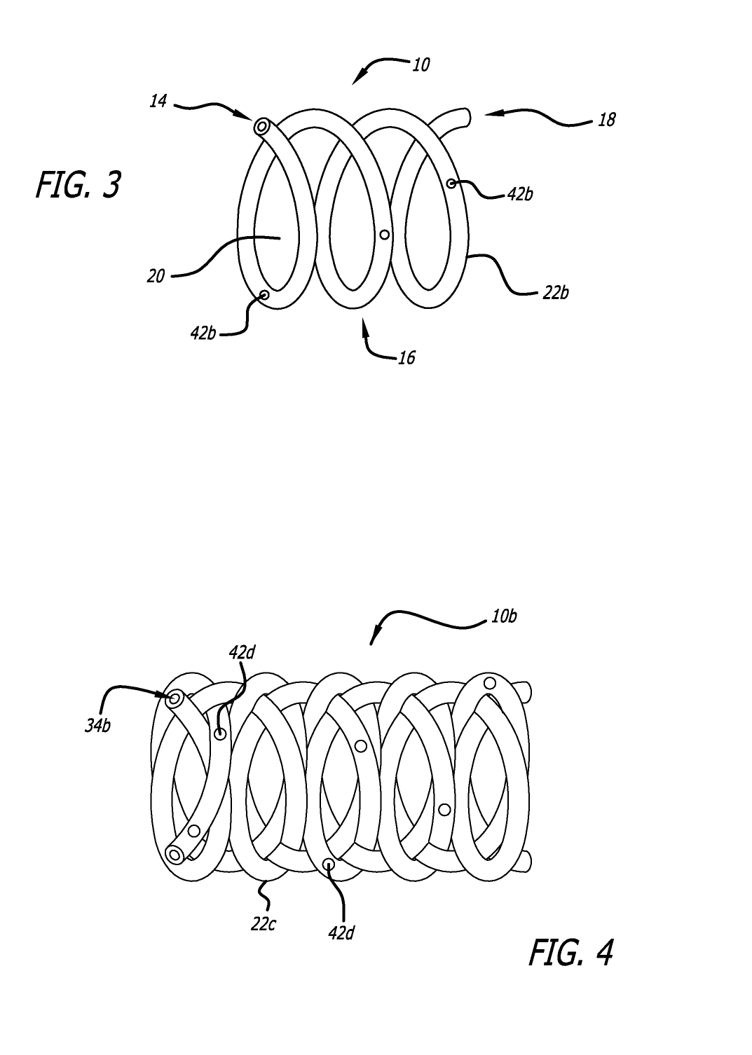

In keeping with this aspect of the present invention, note that in certain embodiments, as shown in FIG. 2, hypotube lumen 34a can be compartmentalized into one or more discrete spaces, for example, compartments 50a, 50b and 50c, to provide areas of the hypotube for different uses. These compartmentalized spaces can be used to more precisely control areas of drug release or can be used to house and release different drugs that cannot co-exist within the same space due to various incompatibilities. Likewise, and as described previously, different compartmentalized areas of a particular hypotube can exhibit similar or different drug release profiles. While FIG. 2 depicts hypotube 22a having three compartments, the present invention includes embodiments of hypotube 22a having more or less compartments. In one embodiment, hypotube 22a contains two compartments. In another embodiment, hypotube 22a contains four compartments. In another embodiment, depicted in FIG. 2b, the hypotube is compartmentalized along its long axis rather than along its azimuthal coordinates into two or more compartments, in a non-limiting example compartments 50d and 50e.

FIG. 3 shows one embodiment of an implantable device according to the present invention. For convenience and brevity, the device depicted in FIG. 3 is a stent. However, it should be noted that other devices or prostheses are also within the scope of the claimed invention. As shown in FIG. 3, stent 10 includes one or more hypotubes 22b that form the body of stent 10. Those skilled in the art will appreciate that hypotubes 22b can be manipulated to form a variety of suitable patterns in forming stent 10, including without limitation, in straight, sinusoidal, coiled, helical, zig-zag, filament type, or V-shaped patterns. Furthermore, a plurality of hypotubes 22b can be formed into stent 10 such that the plurality of hypotubes 22b forms a multiple helix, a braid, a mesh or a woven configuration. As also shown in FIG. 3, stent 10 can be cylindrical or tubular in shape and can have a first end 14, a midsection 16, and a second end 18. Additionally, a hollow channel 20 extends longitudinally through the body structure of the stent 10. The structure of stent 10 allows insertion of stent 10 into a body passageway where stent 10 can physically hold open the passageway by exerting a radially outward-extending force against the walls or inner surface of the passageway. If desired, stent 10 can also expand the opening of the passageway to a diameter greater than the passageway's original diameter and, thereby, increase fluid flow through the passageway. As shown in FIG. 3, hypotube 22b can comprise one or more pores 42b to release drugs contained therein. Alternatively, or in combination, drugs can be released from ends 14 and/or 18, when, e.g., one or both of these ends are not covered or plugged.

Drug release profiles and the particular location of drug release can also be controlled by varying the number, size, and/or placement of pores on a particular hypotube. In one specific, non-limiting example, to reduce or eliminate the incidence of smooth muscle cell proliferation and/or restenosis, the number and/or size of pores can be increased along the channel of the stent for eluting drugs that reduce or prevent cell migration to the channel of the stent. The number and/or size of pores can also be increased at the sites proximal to the walls or inner surface of the passageway for eluting drugs that promote healing of the walls and/or reduce platelet sequestration due to implantation-related injuries.

As previously indicated, those skilled in the art will appreciate that an implantable device according to the present invention (such as a stent) may be manufactured in a variety of sizes, lengths, and diameters (inside diameters as well as outside diameters). A specific choice of size, length, and diameters depends on the anatomy and size of the target passageway, and can vary according to intended procedure and usage.

Referring to FIG. 4, another embodiment of the present invention is described. In this depicted embodiment, hypotubes 22c are configured into a mesh stent 10b in accordance with methods known in the art. In this embodiment, stent 10b comprises a plurality of hypotubes 22c that can be braided in two opposing directions to form the stent 10b. Hypotubes 22c comprise lumen 34b that is in fluid communication with one or more pores 42d to provide localized drug delivery at a treatment site.

In another embodiment, the hypotubes do not have any pores and the drug is delivered by diffusion or a release of drug during degradation of the biodegradable hypotube.

While several embodiments have described the implantable device as a stent, other medical devices would be advantageously formed from the hypotubes according to the teachings of the present invention. Exemplary implantable medical devices include, but are not limited to, stents, stent grafts, urological devices, spinal and orthopedic fixation devices, gastrointestinal implants, neurological implants, cancer drug delivery systems, dental implants, and otolaryngology devices.

A hypotube according to the present invention can be manufactured from a variety of biocompatible materials, such as polymers, that can slowly biodegrade or bioerode over a period of time as a result of its exposure to blood and/or bodily fluid flow. The use of such a biodegradable materials is beneficial in applications where subsequent removal of an implantable device from the patient's body is desired. In one embodiment, the hypotube can be manufactured from such a biocompatible material and one or more pores can exist on the surface of the hypotube upon deployment or/and can appear on the surface as the biocompatible material biodegrades, bioerodes or is bioabsorbed.

Biocompatible, biodegradable materials suitable for manufacturing hypotubes and/or for covering or plugging hypotube pores according to the present invention can include, without limitation, biodegradable metals, metal alloys or polymer. In one embodiment, the biodegradable metal is magnesium or a magnesium alloy. In another embodiment the biodegradable polymer includes, but is not limited to, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen, hyaluronic acid, poly-N-alkylacrylamides, poly depsi-peptide carbonate, polyethylene-oxide based polyesters, and various combinations thereof.

In one embodiment of the present invention, the hypotubes are formed from a biodegradable polymer such as, but not limited to, poly-lactide-co-glycolide or poly-L-lactide-co-caprolactone.

In another embodiment, the pores are plugged with a biodegradable polymer such as, but not limited to poly-lactide-co-glycolide or poly-L-lactide-co-caprolactone.

In another embodiment, a hypotube according to the present invention can be manufactured from a biocompatible, non-erodable polymeric material that does not biodegrade or bioerode over a period of time. Suitable non-erodable polymeric materials include, but are not limited to, polyether sulfone; polyamide; polycarbonate; polypropylene; high molecular weight polyethylene; polydimethylsiolxane, poly(ethylene-vinylacetate); acrylate based polymers or copolymers, e.g., poly(hydroxyethyl methylmethacrylate; polyvinyl pyrrolidinone; fluorinated polymers such as polytetrafluoroethylene; cellulose esters; and the like. Furthermore, the hypotube may also be formed of a semipermeable or microporous material. In non-erodable hypotubes, the materials for covering or plugging hypotube pores can be biodegradable or non-erodable materials as disclosed herein.

Additionally the hypotube can be manufactured from a biodegradable or non-biodegradable material that is porous such that at least one drug loaded within the lumen of the hypotube diffuses across the wall of the hypotube into the vascular environment.

Drugs that are suitable for release from the hypotubes of the present invention include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

In one embodiment of the present invention, the drugs controllably released include, but are not limited to, macrolide antibiotics including FKBP-12 binding agents. Exemplary drugs of this class include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers of the present invention. The entire contents of all of preceding patents and patent applications are herein incorporated by reference for all they teach related to FKBP-12 binding compounds and the derivatives.

The terms “a,” “an,” “the” and similar referents used are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended to better illuminate embodiments according to the invention.

Groupings of alternative elements or embodiments according to the invention disclosed herein are not to be construed as limitations. Each group member may be referred to individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Embodiments of this invention are described herein. Of course, variations on those embodiments will become apparent to those of ordinary skill in the art upon reading the foregoing description unless otherwise indicated herein or otherwise clearly contradicted by context.

Furthermore, numerous references have been made to patents and printed publications throughout this specification. Each of the above cited references and printed publications are individually incorporated herein by reference in their entirety.

What is claimed is:

1. An implantable device for delivering a drug to a treatment site comprising:
a hypotube, said hypotube having a lumen; and
at least one drug disposed within said lumen of said hypotube;
wherein said at least one drug elutes from said hypotube.

2. The implantable device according to claim 1 wherein said at least one drug elutes from said lumen of said hypotube through one or more pores in said hypotube.

3. The implantable device according to claim 1 wherein said at least one drug elutes from said lumen by diffusion through the wall of said hypotube.

4. An implantable device according to claim 2 wherein one or more of said pores are covered or plugged with a biodegradable material.

5. An implantable device according to claim 1 wherein said hypotube is formed from a biodegradable material.

6. An implantable device according to either of claims 4 or 5 wherein said biodegradable material is a material selected from the group consisting of biodegradable metals, metal alloys and polymers.

7. An implantable device according to claim 6 wherein said biodegradable polymer is selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, and combinations thereof.

8. An implantable device according to claim 1 wherein said hypotube is formed from a non-erodable polymeric material selected from the group consisting of polyether sulfone, polyamide, polycarbonate, polypropylene, high molecular weight polyethylene, polydimethylsiolxane, poly(ethylene-vinylacetate), acrylate based polymers or copolymers, polyvinyl pyrrolidinone, fluorinated polymers, and cellulose esters.

9. An implantable device according to claim 1 wherein said implantable device is a stent.

10. An implantable device according to claim 1 wherein said lumen contains at least two compartments.

11. An implantable device according to claim 10 wherein each of said compartments contains different drugs.

12. An implantable device according to claim 10 wherein each of said compartments exhibits different drug release profiles.

13. An implantable device according to claim 2 wherein said hypotube contains more than one pore and said pores are spaced along said hypotube to create different drug release profiles at different portions of said implantable device.

14. The implantable device according to claim 13 wherein the majority of said pores are present on the proximal portion of said implantable device.

15. The implantable device according to claim 13 wherein the majority of said pores are present on the distal portion of said implantable device.

16. An implantable device according to claim 13 wherein said implantable device defines a channel and the majority of said pores are present on the portion of said hypotube contacting said channel.

17. An implantable device according to claim 13 wherein said implantable device defines a channel and the majority of said pores are present on the portion of said hypotube that is generally opposite of the portion of said hypotube contacting said channel.

18. An implantable device according to claim 1 wherein said hypotube is in a configuration selected from the group consisting of a helical configuration, a braided configuration, a mesh configuration and a woven configuration.

19. An implantable device according to claim 1 wherein said implantable device comprises more than one hypotube.

20. An implantable device according to claim 19 wherein said stent comprises two or more hypotubes in a configuration selected from the group consisting of a helical configuration, a braided configuration, a mesh configuration and a woven configuration.

21. An implantable device according to claim 1 wherein said at least one drug is combined with a biocompatible carrier before said drug is disposed within said lumen of said hypotube.

22. An implantable device according to claim 21 wherein said biocompatible carrier comprises a biodegradable material selected from the group consisting of poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, fibrin, fibrinogen, cellulose, starch, collagen, hyaluronic acid, poly-N-alkylacrylamides, poly depsi-peptide carbonate, polyethylene-oxide based polyesters, and combinations thereof.

23. An implantable medical device according to claim 1 wherein said at least one drug is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

24. An implantable medical device according to claim 23 wherein said at least one drug is selected from the group consisting of sirolimus (rapamycin), tacrolimus (FK506), everolimus (certican), temsirolimus (CCI-779) and zotarolimus (ABT-578).

25. An implantable device for delivering a drug to a treatment site comprising:
a biodegradable hypotube, said hypotube having a lumen; and
at least one drug disposed within said lumen of said hypotube;
wherein said at least one drug is released from said lumen upon degradation of said biodegradable hypotube.
